(19) United States
(12) Patent Application Publication
(10) Pub. No.: US 2025/0025053 A1
(43) Pub. Date: Jan. 23, 2025

(54) TWO WAY POINT CONTACTLESS ULTRASOUND SYSTEM

(71) Applicant: Emanuele Raggi, Providence, RI (US)

(72) Inventor: Emanuele Raggi, Providence, RI (US)

(21) Appl. No.: 18/764,591

(22) Filed: Jul. 5, 2024

Related U.S. Application Data
(60) Provisional application No. 63/512,057, filed on Jul. 5, 2023.

(51) Int. Cl. A61B 5/00; G06T 7/00

(52) U.S. Cl. CPC A61B 5/0095; A61B 5/6887; A61B 5/7271; G06T 7/0012; A61B 2562/0219; G06T 2200/04; G06T 2207/10136; G06T 2207/20024; G06T 2207/30096; G06T 2210/41

(57) ABSTRACT

Methods and systems for generating 2D/3D images using dually synchronized pulsed lasers for a contactless ultrasound system. Directing two pulsed wave photoacoustic excitation sources working simultaneously and in synchronization into a desired area distributing acoustic energy into the tissue at the speed of sound. Contrary to a regular hand held ultrasound system, laser-generated ultrasonic waves have the dual advantage of non-contact and non-destructive application without requiring gel, water or electrodes application on the surface of the skin. Two synchronized optical interferometers are applied for detecting ultrasonic waves. The combination and synchronization of two photoacoustic excitation sources, the combination and synchronization of two interferometers combined with external sensors allows for both post-processing and real-time view of images in a very efficient manner. The use of external sensors may be used for image reconstruction and filtering techniques may be applied for 2D/3D image reconstruction and movement compensation.

### CROSS-REFERENCE TO RELATED APPLICATION

This application claims the benefit of the U.S. Provisional Application No. 63/512,057 entitled, “TWO WAY POINT CONTACTLESS ULTRASOUND SYSTEM” filed on Jul. 5, 2023, which is incorporated herein by reference in its entirety.

### TECHNICAL FIELD

Embodiments relate generally to methods and systems to create 2D/3D ultrasound images completely contactless using two laser based ultrasonic sources working simultaneously and in synchronization, two optical interferometers working simultaneously and in synchronization and external sensors.

### STATEMENT REGARDING FEDERALLY SPONSORED RESEARCH

N/A

### BACKGROUND OF THE INVENTION

The present invention relates to systems and methods for generating 2D and 3D ultrasound images without applying any contact to the surface, making the ultrasound system contact-less.

An ultrasound wave occurs at frequencies that are above the bandwidth of human audio capabilities. In terms of imaging, the frequencies cover a wide variety of applications, including, but not limited to, underwater sonar systems, industrial non-destructive testing, near-surface damage assessment, medical diagnostics, and evaluation of acoustic micro-structures.

Regular ultrasonic imaging techniques have long-known benefits. Technological advancements in hand-held probes have been proven to provide better images for soft tissue structures such as the eyes, abdomen, brain, neck, and feet. Conventional techniques for obtaining ultrasound images typically require applying a mechanical probe directly to the patient's body, focusing on the specific area of interest for a particular period of time. The amount of time spent probing is determined by the sonographer's level of experience and by the complexity of the anomaly being investigated.

During an ultrasound, the probe emits an acoustic wave, causing a thermal dilation of tissue that consequently deforms and elasticizes the tissue, generating a returning wave.

These waves ricochet back to the probe, and the time difference between the original emission from the probe and the returning acoustic wave compose the sonogram, which is typically displayed on a monitoring system.

Unlike CT scans, MRIs, or x-rays, ultrasound imaging technologies are relatively low-cost and do not involve the injection of radiation or contrast. They are also portable. Ultrasound technicians and doctors must constantly balance the quality of scans with the hospitals' needs for efficient, high volume scanning to increase healthcare reimbursements.

The use of hand held probes, however, poses problems that current technology is not yet able to overcome. The evidence is clear that overuse injuries brought on by repetitive muscle stresses associated with the performance of ultrasound exams can lead to muscular damage and, in some cases, career-ending injuries. In addition, the hand-held probe may exacerbate existing pain for patients when technicians apply pressure on the point or area under investigation. This is especially prominent during a prolonged pressure application to a painful area.

Patients may also move during the procedure due to discomfort or pain, and this can interfere with the quality of the scan, corrupt the ultrasonic reading, or elongate the duration of the exam. If patients are in extreme pain or cannot be still during a scan, the hospital may have to reschedule a session, since efficiency and patient turnover must remain high in the current profit structure.

A patient's body type and size also impact the quality of an ultrasound. With overweight and obese patients, the ultrasound wave has to pass through more fat tissue before reaching the main site, sometimes causing an unclear reading. In addition, internal gas can also interfere with the accuracy of a reading, requiring a longer session. In these cases, the sonographer must apply a great deal of pressure to the site.

In order to produce an accurate reading using current ultrasound technology, the system must always have a clear understanding of where the probe is in space. Spatial resolution is often a major obstacle with handheld devices. Accurate readings require the system to have a continuous record of the angular velocity, linear velocity, roll, pitch, and yaw. While current technology offers spherical probe transducers, which concentrate the beam, there is still too much room for human error. For example, if the technician's hand trembles or if there is inconsistency in the amount of pressure they apply, this can compromise the integrity of the reading. In addition, if the sonographer did not apply enough gel to create the ideal electrodynamic balance, the returning wave could be flawed and lead to an inaccurate scan.

There is currently a significant amount of scientific research exploring image registration and feature matching when performing image reconstruction. Before an exam is even performed, all sensors must be calibrated so that images can be properly aligned during reconstruction. Researchers have also investigated the use of point cloud and motion compensation techniques to perform more accurate image reconstruction.

Based on the limitations of current technology, there is a need for more advanced point registration techniques and better 2D and 3D image reconstruction procedures. More advanced technology would lead to stronger decision making by physicians and higher quality diagnostic imagery. This is especially important because ultrasound technicians and doctors are constantly under the pressure of daily patient turnover and it is critical that the highest possible image quality is available when making high stakes and time-sensitive medical decisions.

Robotic-assisted surgical ablation currently utilizes ultrasounds to guide and support the procedures in real time. Because sterility is critical, there is an extensive and lengthy hospital procedure for ensuring that the probe is properly cleaned and prepared. This process must be repeated for each procedure in order to minimize the risk of infection, which is a drain on hospital resources.

Physician-led procedures, for example, liver biopsies or tumor ablation, that already utilize handheld ultrasound would also benefit from Artificial Intelligence-guided ultrasounds in order to reduce patient risk and improve outcomes.

Research and experimentation into pulse or continuous lasers to be used as contactless ultrasonic devices is in the initial stages. Some studies have proven that contactless ultrasound techniques can be a useful tool for both humans and animals. Eye-safe techniques are already being used to treat optical problems such as myopia and are considered safe at a particular exposure. Wireless portable ultrasound probes are already in use and present a great advantage in terms of portability and field operations. They do not, however, eliminate the need to physically touch the patient nor do they limit sonographers' stress injuries. This demonstrates a need for developing novel techniques in this field that allow for acoustic waves to enter soft tissue, skin, and organs without the need for physical contact with the patient.

### SUMMARY OF THE INVENTION

The following presents a simplified summary of the innovation in order to provide a basic understanding of some aspects of the invention. This summary is not an extensive overview of the invention. It is intended to neither identify key or critical elements of the invention nor delineate the scope of the invention. Its sole purpose is to present some concepts of the invention in a simplified form as a prelude to the more detailed description that is presented later. In order to overcome the aforementioned limitations in current ultrasound technology, the present invention utilizes simultaneous and synchronous laser-generated ultrasound waves combined with the use of exteroceptive sensors and filtering techniques.

The application of laser ultrasonics for clinical diagnostic assessment of the internal structure of tissues carries significant advantages over current models. A pulse laser, or Q-switched laser, may be used for internal localization and detection of features or soft tissues by emitting a photoacoustic ultrasonic wave toward the patient. The return wave is represented by a coherent summation of waves that present themselves on the patient's skin and are analyzed with an optical tool such as an interferometer. When the returning wave ricochets back to the surface it presents itself as a vibration at that specific point. That vibration corresponds to the maximum vibration detected at that point using the optical interferometer. The maximum vibrational point is then captured with the interferometer and given the proper xyz coordinate which will be properly assigned to a grid heatmap. The grid heatmap displays magnitude as color in a two-dimensional matrix. Each dimension represents a category of trait and the color represents the magnitude of specific measurement on the combined traits from each of the two categories. One dimension represents the length of the scanned area and the other dimension represents the width of the scanned area, and the value measured is the depth. This grid heat map would show how depth changes across different locations of the scanned area while the simultaneous and synchronous Q-switched laser device is moving according to a predefined area.

One embodiment of the present invention provides a method for generating 2D/3D ultrasound images of a patient using a single laser-based ultrasonic source. Unlike conventional ultrasonic efforts to establish the internal structure of soft tissues or organs, laser generation techniques, being contactless, do not require any specific preparation of the skin. The method includes generating ultrasonic acoustic waves using pulsed wave lasers. The beam of the excitation source is focused on a specific area of interest of the body and emits ultrasonic waves. The ultrasonic excitation source may include a lens apparatus able to direct and guide the beam.

Due to backscattering, returning acoustic waves are detected via interferometric systems. Detected waveforms on the skin of the patient are subsequently analyzed and processed to evaluate the internal structure. Exteroceptive sensors, such as a colored camera, a black and white camera, an Inertial Measurement Unit (IMU), a GPS, or an RTK GPS antenna, are used in combination with a robotic platform. Since a patient may move during the ultrasound session, the sonographer or the doctor can highlight specific points on the skin of the patient with a highlighter allowing the cameras to feature-detect those points and have a specific search area that keeps the reference frame stable.

Detected waveforms on the skin of the patient are subsequently analyzed by a processor which provides ultrasound images that may evaluate the internal structure. Ultimately, the processing step is directly connected to a data acquisition system that may be able to record the necessary data of all exteroceptive sensors.

Another embodiment of the present invention provides a method for generating 2D/3D ultrasound images of a patient using two laser-based ultrasonic sources simultaneously and in synchronization. The method includes generating ultrasonic acoustics from two different sources using pulsed lasers. The beams of the excitation sources are focused on a specific area of interest in the body and emit ultrasonic waves. The pulses of the lasers alternate each other. By way of example: Q-Switched laser A sends a pulse a time 0 at position 0 so the first wave can penetrate the surface of the skin and arrive at the preferred depth. Q-Switched laser B sends a pulse, for example, 10 nanoseconds later a time 1 at position 1 so the second wave can penetrate the surface of the skin and arrive at the preferred depth. The interval between the two pulses is when the maximum surface wave vibration is detected from the external interferometer. So in a continuous way, the system translates at extremely low speed while it performs the scan alternating the two pulses from the two different lasers. Additionally, in a continuous way, the system rotates at extremely low speed while it performs the scan alternating the two pulses from the two different lasers.

An additional embodiment of the present invention provides a method for generating 2D/3D ultrasound images of a patient. The method stores data points from the lasers and all exteroceptive sensor positions and orientations in an information log upon indication of the sonographer or the doctor. The stored data points are used to build a list of vector positions and orientations which can then be used to play-back the recorded session, train sonographers or doctors or further studies.

Still another embodiment of the present invention provides a method for generating 2D/3D ultrasound images of a patient by displaying an ultrasound view of an anatomic structure derived from two laser-based ultrasonic sources working simultaneously and in synchronization. The system works by applying the concept of superposition to overlap layers of images taken from the various cameras and lasers, aligning with the position and orientation of the exteroceptive sensors in order to accurately reconstruct the images. This embodiment can be divided into the following steps: a) since a patient may move during the ultrasound session, the sonographer or the doctor can highlight specific points on the skin of the patient with a highlighter allowing the cameras to feature-detect those points and have a specific search area that keeps the reference frame stable; b) the two laser-based ultrasonic sources working simultaneously and in synchronization start emitting pulses alternating laser A and laser B on the specific area mentioned at point a) allowing for surface wave maximum detection; c) the interferometer detects those maximum vibrational waves within the area and assign xyz location to every point; d) all those points are combined together to create a heat grid map at that specific depth; e) laser A and laser B are regulated to have a higher or lower penetration rate so it is possible to create another layer (another grid map) at a different depth but within the same area mentioned at point a); f) repeat process e) for several depth so that the sonographer can form different layers at different depths of the same search area; g) interpolate the different layers in order to form a 3D image completely contactless.

Knowledge of the acoustic wavelengths between different structures of the body allows for internal structures to be reconstructed, therefore between 1400 nanometers (nm) and 1600 nanometers (nm) are the preferred emitting ranges of the pulsed lasers.

Additionally, compensation techniques used for image reconstruction may be derived from non-parametric filtering techniques, included but not limited to, histogram filters, particle filters and gaussian probability hypothesis density filters.

Additional features and advantages of the various aspects of the present invention become apparent from the following description of its preferred embodiments. The description provided should be taken in consideration with the accompanying drawings. The aforementioned embodiments do not represent the full scope of the invention. The references following the description of the preferred embodiments are made thereof to the claims and herein for interpreting the scope of the present invention.

Other objects, features and advantages of the invention shall become apparent as the description thereof proceeds when considered in connection with the accompanying illustrative drawings.

### BRIEF DESCRIPTION OF THE DRAWINGS

The novel features that are characteristic of the present invention are set forth in the appended claims. However, the invention's preferred embodiments, together with further objects and attendant advantages, will be best understood by reference to the following detailed description taken in connection with the accompanying Figures:

FIG. 1 is an image showing a system configured to implement the present invention for 3D reconstruction of images.

FIG. 2 is an image showing a portable system using two remote excitation sources configured to implement the present invention for 3D reconstruction of images.

FIG. 3 is an image showing a system configured to implement the present invention for 3D reconstruction of images.

FIG. 4 is an image showing a portable system using two remote excitation sources configured to implement the present invention for 3D reconstruction of images.

FIG. 5 is a flow chart explaining the post-processing procedure and image reconstruction for two laser sources and two interferometers with an additional correction step.

FIG. 6 is a flow chart explaining the post-processing procedure and image reconstruction for two laser sources and two interferometers.

FIG. 7 is a flow chart explaining the real-time processing procedure and image reconstruction for two laser sources and two interferometers.

FIG. 8 is a flow chart explaining the real-time processing procedure and image reconstruction for two laser sources and two interferometers with an additional correction step.

### DETAILED DESCRIPTION OF THE INVENTION

The subject innovation is now described with reference to the drawings, wherein like reference numerals are used to refer to like elements throughout. In the following description, for purposes of explanation, numerous specific details are set forth in order to provide a thorough understanding of the present invention. It may be evident, however, that the present invention may be practiced without these specific details. In other instances, well-known structures and devices are shown in block diagram form in order to facilitate describing the present invention.

For convenience, the meaning of some terms and phrases used in the specification, examples, and appended claims, are listed below. Unless stated otherwise or implicit from context, these terms and phrases have the meanings below. These definitions are to aid in describing particular embodiments and are not intended to limit the claimed invention. Unless otherwise defined, all technical and scientific terms have the same meaning as commonly understood by one of ordinary skills in the art to which this invention belongs. For any apparent discrepancy between the meaning of a term in the art and a definition provided in this specification, the meaning provided in this specification shall prevail.

“Data Acquisition System (DAS)” has the electronic hardware art-defined meaning. A DAS for the context of this patent is intended to be an oscilloscope which is able to process data originating from the backscattering of ultrasonic waveforms on the surface of the tissue.

“Data Log (DL)” has the database art-defined meaning. A DL for the context of this patent is intended to be a storage system such as a database, where all information from external sensors are continuously stored in real-time. Such information is, for example, displacement, position, velocity and acceleration.

“External Sensors (ES)” has the hardware art-defined meaning. The external sensors for the context of this patent are defined as the two lasers, the interferometric receiving system (receiver device), the GPS, the color camera, the black and white camera and the stepping motors. There is a 3-axis IMU mounted on each of these sensors.

“External Visualizer (EV)” has the hardware art-defined meaning. The EV is intended as a regular computer monitor. A 3D visualizer is also a computer monitor where to show the 3D image. For the context of this patent external visualizer and 3D visualizer are intended as regular computer monitor and these terms will be used interchangeably.

“Filtering Techniques (FT)” has the mathematical robotic art-defined meaning. FT processes are algorithms aimed at finding patterns in historical and current data to extend them into future predictions, providing insights into what might happen next. Filtering techniques can be divided into parametric and non-parametric as explained below.

“Global Positioning System (GPS)” has the mathematical robotic art-defined meaning. GPS is a radio navigation system that allows land, sea, and airborne users to determine their exact location, velocity, and time 24 hours a day, in all conditions, in every instant of time. A GPS system can be considered as the absolute reference frame system from which local relative reference frames refer to while in movement.

“Grid Heatmap (GH)” has the mathematical art-defined meaning. The GH displays magnitude as color in a two-dimensional matrix. Each dimension represents a category of trait and the color represents the magnitude of specific measurement on the combined traits from each of the two categories. One dimension represents the length of the scanned area and the other dimension represents the width of the scanned area, and the value measured is the depth. This grid heat map would show how depth changes across different locations of the scanned area under investigation. The variation in color may be by hue or intensity, giving obvious visual cues to the user about how the phenomenon varies over space.

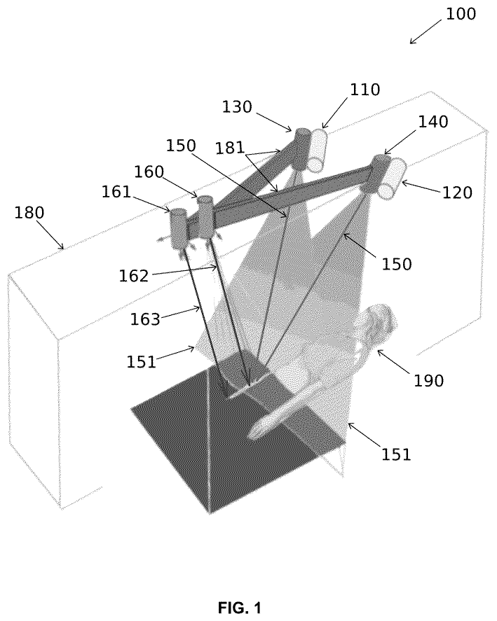

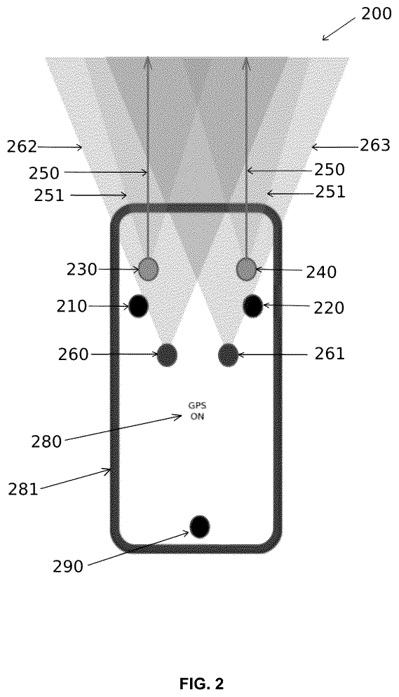

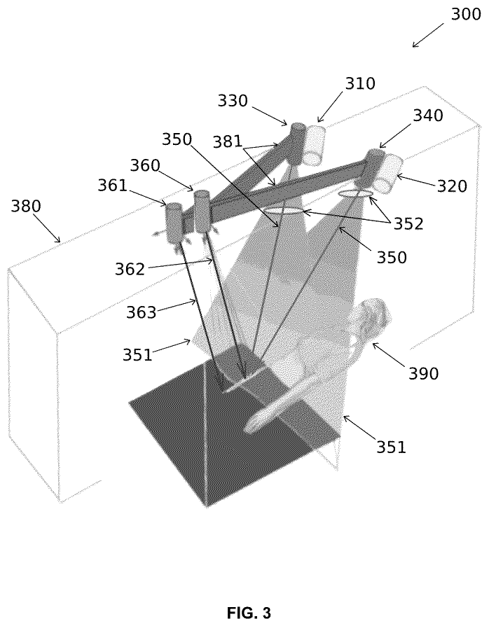

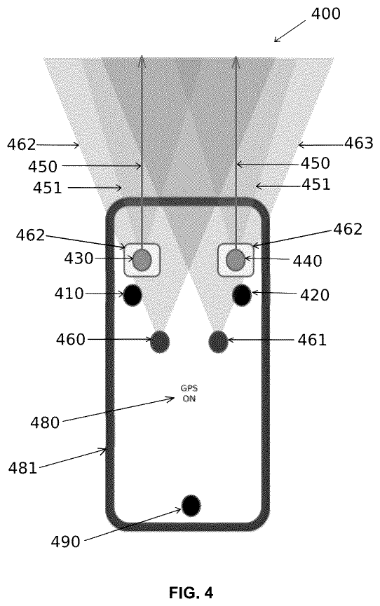

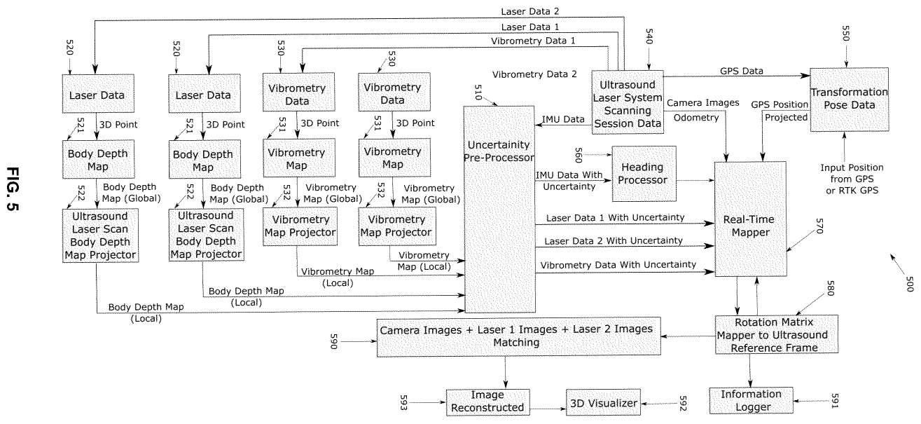

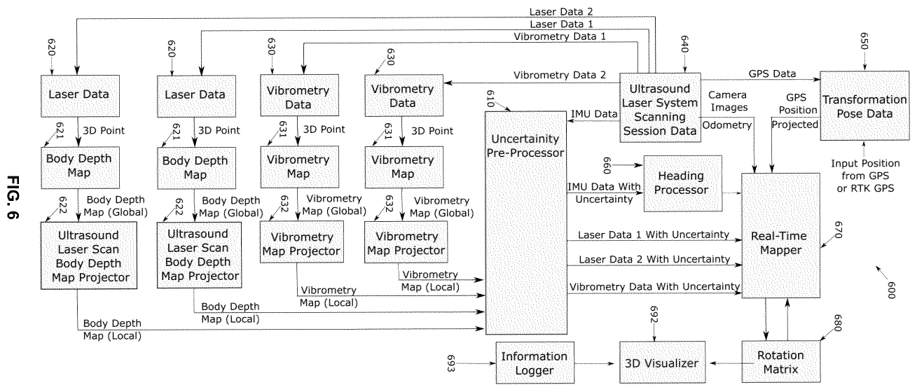

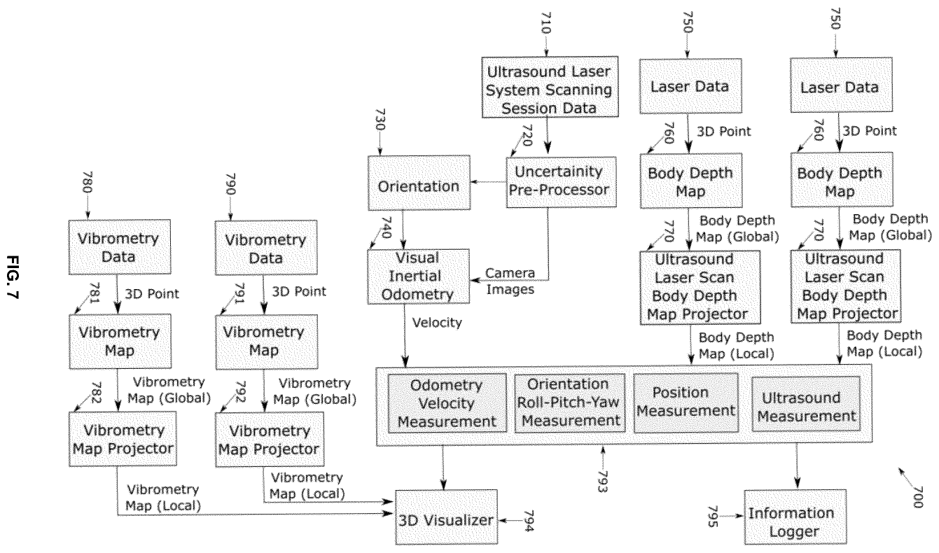

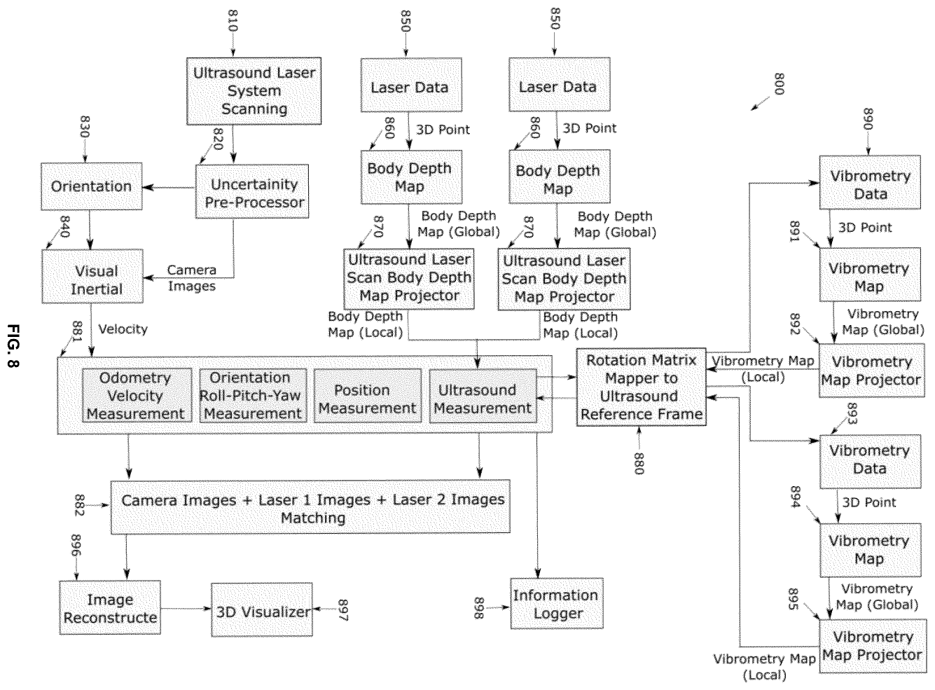

“Inertial Measurement Unit (IMU)” has the mathematical robotic art-defined meaning. IMUs are inertial sensors commonly used in robotics motion, terrain compensation, platform stabilization, antenna and camera pointing applications. An individual IMU can sense a measurement along or about a single axis. To provide a three-dimensional solution, three individual IMUs must be mounted together into an orthogonal cluster. This set of IMUs mounted is commonly referred to as a 3-axis inertial sensor, as the sensor can provide one measurement along each of the three axes. Therefore, in this patent every sensor listed (colored camera, black and white camera, GPS, or RTK GPS antenna, Q-switched laser(s) and interferometer) will be equipped with a 3-axis IMU so that measurement on each direction (roll-pitch-yaw) is available at any time and in every instant; specifically: FIG. 1, FIG. 3, FIG. 5, FIG. 7, FIG. 9 and FIG. 11. Additionally when referring to the 3-axis IMU mounted on a portable device such as FIG. 2, FIG. 4, FIG. 6, FIG. 8, FIG. 10 and FIG. 12, one single 3-axis IMU sensor will be mounted on the portable device.

“Information Logger (IL)” has the database art-defined meaning. An IL for the context of this patent is intended to be a storage system such as a database, where all information from external sensors are continuously stored. Such information are, for example, displacement, position, velocity and acceleration.

“Kalman Filter” has the mathematical art-defined meaning. The Kalman Filter is an optimal estimator defined as a set of mathematical equations that provides a recursive computational methodology for estimating the state of a discrete process from measurements that are typically noisy, while providing an estimate of the uncertainty of the estimates. Kalman Filter uses linear approximations of the state and measurement models to generate a state estimate given a prior state and associated uncertainties.

“Laser Stereometry (LS)” has the mathematical robotic art-defined meaning. A laser stereo image contains two views of a scene side by side. One laser view is intended for the left eye and the other laser view for the right eye.

“Non-Parametric Filter” has the mathematical robotic art-defined meaning. Non-parametric filters do not rely on any specific parameter settings and therefore tend to produce more accurate results. Non-parametric filters approximate posteriors by a finite number of values, each roughly corresponding to a region in state space. Examples of non-parametric filters are: histogram filter, particle filter, PHD filter, gaussian mixture PHD filter.

“Odometry” has the mathematical robotic art-defined meaning. Odometry is a common method of determining an object's motion from the way in which subsequent images overlap.

“Particle Filter (PF)” has the mathematical robotic art-defined meaning. The PF is a non-parametric solution to the Bayes Filter which uses a set of samples or “particles”. Each one of these particles can be seen as the possibility of an object being at that position at that time. PF is divided into three main steps. Prediction or state transition model, where the particle's position is predicted according to external sensors. Weighting or measurement model, where particles are weighted according to their likelihood with data provided by external sensors, and finally, resample, where particles with small weights are discarded.

“Processing System (PS)” has the hardware art-defined meaning. PS or Processor System is a computer such as a laptop or tower desktop able to process data in real-time or post-processing or pre-processing originated from a scanning session.

“Robotic Platform (RP)” has the robotic art-defined meaning. A RP is a set of open-source software frameworks, libraries, and tools to create applications for robots such as the Robotic Operating System (ROS). A RP provides a range of features that come with a standard operating system such as hardware abstraction and sensor integration package management. Additionally, a RP allows the user to develop customized software and processes to enhance existing features of sensors. All sensors listed in this patent can be integrated into a RP such as IMUs, Interferometers, Q-Switched Lasers, Cameras (colored and black and white) and stepping motors.

“Posterior” has the statistics art-defined meaning. A posterior, also known as posterior probability, in Bayesian statistics, is the revised or updated probability of an event occurring after taking into consideration new historical information in order to predict what might happen next. The posterior probability is calculated by updating the prior probability using Bayes' theorem. The posterior probability is the probability of event A occurring given that event B has occurred.

“Prior” has the statistics art-defined meaning. A Prior also known as prior probability, in Bayesian statistics, is the probability of an event occurring before new (posterior) data is collected.

“Real-Time Mapper (RTM)” has the mathematical robotic art-defined meaning. In order to properly position images in real-time on the correct reference frame a rotation matrix transformation from Real-Time Mapper to Ultrasound Reference frame is needed. The RTM is in charge of correctly showing the projection of the heat grid map in the real-time reference frames while the system is scanning the patient.

“Stereo Images (SI)” has the mathematical robotic art-defined meaning. A stereo image contains two views of a scene side by side. One of the views is intended for the left eye and the other for the right eye. A stereo pair of images taken at each view's location is used to find distance. Then, the image from one camera is registered to the same camera's previous image, correcting rotation and scale differences. This registration is used to find the area's motion and speed between imaging positions.

“Ultrasound Laser System Scanning Session Data (ULSSSD)” has the robotic art-defined meaning. The ULSSSD is the main container of the recorded session. All necessary data such as laser sources, external sensors such as cameras, 3-axis IMU, vibrometric sensor and a GPS, or an RTK GPS antenna system are recorded in the ULSSSD. ULSSSD works both real-time and post-processing.

“Vibrometric Sensor (VS)” has the robotic art-defined meaning. For the context of this patent a VS or interferometer have the same meaning. These instruments are able to optically detect surface vibrations. Therefore, these terms will be used interchangeably in this patent.

“Visual Inertial Odometry (VIO)” has the mathematical robotic art-defined meaning. VIO is a common method of determining an object's motion from the way in which subsequent images overlap.

### Example 1: Simultaneous and Synchronized Q-Switched Pulsed Lasers, Colored Camera, Black and White Camera, Two Interferometers, GPS and 3-Axis IMU

FIG. 1 illustrates an exemplary system 100 configured to implement the present invention for 3D reconstruction of images with two excitation sources 130 and 140 configured to be implemented into the system working simultaneously and in synchronization. Additionally there are external sensors such as a colored camera 110 and a black and white camera 120, the 3-axis IMU (integrated in the structure 180), the GPS (integrated in the structure 180), or RTK GPS antenna, for real-time positioning and the two interferometers 160 and 161. 151 simply represents the field of view of the lasers.

Images taken from a colored camera 110 and a black and white camera 120 are used to form stereo images and provide a 3D external shape of the patient 190. Patient's movement is compensated via real-time visual inertial odometry. Stereo images are used during the external reconstruction of the human shape providing a state of the art measured image. Knowing the position of the two cameras allows precise measurement of the patient 190. The 3-axis IMU used for real-time position and orientation integrated in the structure 180 is not visible but is present. Additionally a 3-axis IMU is integrated in the GPS (not visible but is present in the structure 180), or an RTK GPS antenna, in every Q-Switched laser 130 and 140, in the colored camera 110, in the black and white camera 120 and in the two interferometers 160 and 161. Since a patient may move during the ultrasound session, the sonographer or the doctor can highlight specific points on the skin of the patient with a highlighter allowing the cameras to feature-detect those points and have a specific search area box.

Two Q-switched pulsed lasers 130 and 140 emit an ultrasonic wave on top of the human body, or on the preferred part, that will propagate in the internal part of the body. Through a backscatter coherent summation of the return waves up to the skin of the patient it is possible to detect such displacement via two interferometric systems 160 and 161. Lasers 130 and 140 operate simultaneously and in synchronization with the other external sensors.

In a hospital setting system 100 fits all around the patient and all the external sensors such as Q-Switched pulsed lasers, colored camera, black and white camera, interferometers, GPS are present. All the sensors can be fixed while the platform 181 moves (translates) accordingly after the input of the doctor's or the sonographer's about the part to scan. Alternatively, the sensors remain fixed and the system translates and rotates accordingly on top of the search area decided by the doctor by way of stepping motors.

The ultrasonic wave is emitted in the wavelength where the absorption is preferred by the human body and its internal tissues or organs in order to have an optimal response.

The interferometers 160 and 161 detects those maximum vibrational waves within the area the doctor's or the sonographer's decided to scan and assign xyz location to every point. All those points are combined together to create a heat grid map at that specific depth.

162 and 163 simply represent the two interferometers observing the maximum vibration detected on the skin at a specific point. Returning waves on the skin outside of the field of view of the interferometer will only give a partial or incomplete reading. Therefore, it is important that the field of view of the two lasers 151 and the field of view of the interferometer overlap for optimal detection.

The lasers are regulated to have a higher or lower penetration rate so it is possible to create another layer (another grid map) at a different depth but within the same investigational area mentioned above. Repeating the scanning process for several depths will provide the sonographer with different layers of the preferred area of investigation. At this point it is possible to simply interpolate the different layers in order to form a 3D image completely contactless.

Between two different laser pulses from the two laser sources is the moment where the maximum vibrational point is optically observed by the interferometer on the skin. That point is assigned specific xyz coordinates that will constitute a heat grid map. The laser beams may move, or be guided through the lens apparatus, on top of the search area established by the doctor or the sonographer and move according to specific patterns such as lawn-mower.

Additional real-time grid map construction can be built using filtering techniques, specifically using non-parametric filters such as particle filter techniques. As an additional note, the Q-Switched lasers can be used both with the same wavelength and obtain a vibrational layer at a specific depth preferred by the doctor, or one Q-Switched laser with wavelength A and the second Q-Switched laser with wavelength B. This will allow different penetration depth at the same time and the possibility to detect with the dually synchronized interferometers different vibrational patterns from different depth. This is also dependent on the preferred choice from the doctor's side. Therefore, the prediction technique with the particle filter can be used simultaneously both with Q-Switched lasers of the same wavelength and with Q-Switched lasers of different wavelengths to have real-time heatmaps images. Those images can finally be interpolated to extract a final 3D image.

### Example 2: Portable System

In case of an exemplary handheld device as shown in FIG. 2, the system 200 and the same sensors are used such as a colored camera 210 and a black and white camera 220, the 3-axis IMU 290, the GPS 280, or RTK GPS antenna, for real-time positioning, the Q-switched lasers 230 and 240 and the interferometers 260 and 261. 251 simply represents the field of view of the laser.

The 3-axis IMU 290 is able to provide a precise orientation together with linear and angular velocities being able to detect the smallest movement in roll, pitch and yaw.

The 3-axis IMU 290 integrates multi-axis, accelerometers and gyroscopes altogether to provide an object's orientation in space. Together the emitting laser beam 250 and the external interferometer 260 and 261 are able to detect the receiving ultrasonic waves as a result of coherent summation on the skin. Returning waves on the skin outside of the field of view of the interferometer will only give a partial or incomplete reading. Therefore, it is important that the field of view of the laser 251 and the field of view of the interferometer 262 and 263 overlap for optimal detection. The 3D images are visualized on a portable device 281 remote from the patient that enables real-time functioning. The interferometer 260 and 261 detects those maximum vibrational waves within the search area established by the sonographer or the doctor and assign xyz location to every vibrational point detected. All those points are combined together to create a heat grid map at that specific depth. The laser is regulated to have a higher or lower penetration rate so it is possible to create another layer (another grid map) at a different depth but within the same investigational area mentioned above. Repeating the scanning process for several depths will provide the sonographer with different layers of the preferred area of investigation. At this point it is possible to simply interpolate the different layers in order to form a 3D image completely contactless.

Between two different laser pulses is the moment where the maximum vibrational point is optically observed by the interferometer on the skin. That point is assigned specific xyz coordinates that will constitute a heat grid map. The laser beam may move on top of the search area established by the doctor or the sonographer and move according to specific patterns such as lawn-mower.

Additional real-time grid map construction can be built using filtering techniques, specifically using non-parametric filters such as particle filter techniques. As an additional note, the Q-Switched lasers can be used both with the same wavelength and obtain a vibrational layer at a specific depth preferred by the doctor, or one Q-Switched laser with wavelength A and the second Q-Switched laser with wavelength B. This will allow different penetration depth at the same time and the possibility to detect with the dually synchronized interferometers different vibrational patterns from different depth. This is also dependent on the preferred choice from the doctor's side. Therefore, the prediction technique with the particle filter can be used simultaneously both with Q-Switched lasers of the same wavelength and with Q-Switched lasers of different wavelengths to have real-time heatmaps images. Those images can finally be interpolated to extract a final 3D image.

### Example 3: Simultaneous and Synchronized Q-Switched Pulsed Lasers, Colored Camera, Black and White Camera, Two Interferometers, GPS, 3-Axis IMU and Lens Apparatus

FIG. 3 illustrates an exemplary system 300 configured to implement the present invention for 3D reconstruction of images with two excitation sources 330 and 340 configured to be implemented into the system working simultaneously and in synchronization. Additionally there is a lens apparatus 352 able to guide the ultrasonic emitting beams 350 together with the external sensors such as a colored camera 310 and a black and white camera 320, the 3-axis IMU (integrated in the structure 380), the GPS (integrated in the structure 380), or RTK GPS antenna, for real-time positioning and the two interferometers 360 and 361. 351 simply represents the field of view of the laser.

Images taken from a colored camera 310 and a black and white camera 320 are used to form stereo images and provide a 3D external shape of the patient 390. Patient's movement is compensated via real-time visual inertial odometry. Stereo images are used during the external reconstruction of the human shape providing a state of the art measured image. Knowing the position of the two cameras allows precise measurement of the patient 390. The 3-axis IMU used for real-time position and orientation integrated in the structure 380 is not visible but is present. Additionally a 3-axis IMU is integrated in the GPS (not visible but is present in the structure 380), or an RTK GPS antenna, in every Q-Switched laser 330 and 340, in the colored camera 310, in the black and white camera 320 and in the two interferometers 360 and 361. Since a patient may move during the ultrasound session, the sonographer or the doctor can highlight specific points on the skin of the patient with a highlighter allowing the cameras to feature-detect those points and have a specific search area box.

Two Q-switched pulsed lasers 330 and 340 emit an ultrasonic wave on top of the human body, or on the preferred part, that will propagate in the internal part of the body. Through a backscatter coherent summation of the return waves up to the skin of the patient it is possible to detect such displacement via two interferometric systems 360 and 361. Lasers 330 and 340 operate simultaneously and in synchronization with the other external sensors.

In a hospital setting system 300 fits all around the patient and all the external sensors such as Q-Switched pulsed lasers, colored camera, black and white camera, interferometers, GPS are present. All the sensors can be fixed while the platform 381 moves (translates) accordingly after the input of the doctor's or the sonographer's about the part to scan. Alternatively, the sensors remain fixed and the system translates and rotates accordingly on top of the search area decided by the doctor and the lens apparatus will guide the beam by way of stepping motors.

The ultrasonic wave is emitted in the wavelength where the absorption is preferred by the human body and its internal tissues or organs in order to have an optimal response. The interferometers 360 and 361 detects those maximum vibrational waves within the area the doctor's or the sonographer's decided to scan and assign xyz location to every point. All those points are combined together to create a heat grid map at that specific depth.

362 and 363 simply represent the two interferometers observing the maximum vibration detected on the skin at a specific point. Returning waves on the skin outside of the field of view of the interferometer will only give a partial or incomplete reading. Therefore, it is important that the field of view of the two lasers 351 and the field of view of the interferometer overlap for optimal detection.

The lasers are regulated to have a higher or lower penetration rate so it is possible to create another layer (another grid map) at a different depth but within the same investigational area mentioned above. Repeating the scanning process for several depths will provide the sonographer with different layers of the preferred area of investigation. At this point it is possible to simply interpolate the different layers in order to form a 3D image completely contactless.

The lens apparatus 352 is able to guide the ultrasonic emitting beam. The lens apparatus 352 can also be used to guide the laser beams in a specific area of the body.

Between two different laser pulses from the two laser sources is the moment where the maximum vibrational point is optically observed by the interferometer on the skin. That point is assigned specific xyz coordinates that will constitute a heat grid map. The laser beams may move, or be guided through the lens apparatus, on top of the search area established by the doctor or the sonographer and move according to specific patterns such as lawn-mower.

Additional real-time grid map construction can be built using filtering techniques, specifically using non-parametric filters such as particle filter techniques. As an additional note, the Q-Switched lasers can be used both with the same wavelength and obtain a vibrational layer at a specific depth preferred by the doctor, or one Q-Switched laser with wavelength A and the second Q-Switched laser with wavelength B. This will allow different penetration depth at the same time and the possibility to detect with the dually synchronized interferometers different vibrational patterns from different depth. This is also dependent on the preferred choice from the doctor's side. Therefore, the prediction technique with the particle filter can be used simultaneously both with Q-Switched lasers of the same wavelength and with Q-Switched lasers of different wavelengths to have real-time heatmaps images. Those images can finally be interpolated to extract a final 3D image.

### Example 4: Portable System

In case of an exemplary handheld device as shown in FIG. 4, the system 400 and the same sensors are used such as a colored camera 410 and a black and white camera 420, the 3-axis IMU 490, GPS 480, or RTK GPS antenna, for real-time positioning, the Q-switched laser 430 and 440 with their lens apparatuses 462 and the interferometers 460 and 461. 451 simply represents the field of view of the lasers.

The 3-axis IMU 490 is able to provide a precise orientation together with linear and angular velocities being able to detect the smallest movement in roll, pitch and yaw.

The 3-axis IMU 490 integrates multi-axis, accelerometers and gyroscopes altogether to provide an object's orientation in space. Together the emitting laser beam 450 and the external interferometer 460 and 461 are able to detect the receiving ultrasonic waves as a result of coherent summation on the skin. The 3D images are visualized on a portable device 481 remote from the patient that enables real-time functioning. The interferometers 460 and 461 detects those maximum vibrational waves within the search area established by the sonographer or the doctor and assign xyz location to every vibrational point detected. All those points are combined together to create a heat grid map at that specific depth. The lasers are regulated to have a higher or lower penetration rate so it is possible to create another layer (another grid map) at a different depth but within the same investigational area mentioned above. Repeating the scanning process for several depths will provide the sonographer with different layers of the preferred area of investigation. At this point it is possible to simply interpolate the different layers in order to form a 3D image completely contactless.

The lens apparatus 462 is able to guide the two ultrasonic emitting beams 451. The lens apparatus 462 can also be used to guide the laser beams 451 in a specific area of the body.

Between two different laser pulses from the two laser sources is the moment where the maximum vibrational point is optically observed by the interferometer on the skin. That point is assigned specific xyz coordinates that will constitute a heat grid map. The laser beams may move, or be guided through the lens apparatus, on top of the search area established by the doctor or the sonographer and move according to specific patterns such as lawn-mower.

Additional real-time grid map construction can be built using filtering techniques, specifically using non-parametric filters such as particle filter techniques. As an additional note, the Q-Switched lasers can be used both with the same wavelength and obtain a vibrational layer at a specific depth preferred by the doctor, or one Q-Switched laser with wavelength A and the second Q-Switched laser with wavelength B. This will allow different penetration depth at the same time and the possibility to detect with the dually synchronized interferometers different vibrational patterns from different depth. This is also dependent on the preferred choice from the doctor's side. Therefore, the prediction technique with the particle filter can be used simultaneously both with Q-Switched lasers of the same wavelength and with Q-Switched lasers of different wavelengths to have real-time heatmaps images. Those images can finally be interpolated to extract a final 3D image.

### Example 5: Flow Chart Explaining the Post-Processing Procedure and Image Reconstruction for Two Sources Q-Switched Pulsed Laser Working Simultaneously and in Synchronization, Colored Camera, Black and White Camera, Two Interferometers Working Simultaneously and in Synchronization, GPS and 3-Axis IMU

Referring to FIG. 5, a flow chart setting forth the most important steps for post-processing and generating laser images is provided. To start the post-processing loop and generate 2D/3D laser images (i.e. playing back an ultrasound session using a Robotic Platform such as ROS) related to the architecture shown in FIG. 1, FIG. 2, FIG. 3 or FIG. 4, the user needs to start from where all the data from the scanning session is collected, which is the scanning session data block 540. The ultrasound lasers system scanning session data block 540 is the main container of the recorded ultrasound session. Such data are, for example, displacement, position, velocity and acceleration of every sensor and even monitoring the movements of the stepper motors. The presence of the GPS makes sure that there is an absolute reference frame from which all the other local reference frames given from the other sensors relate to while the system translates or roto-translate around the patient.

All necessary data coming from laser sources, external sensors such as color camera, black and white camera, 3-axis IMU, interferometers and a GPS, or an RTK GPS antenna system, 550 are characterized by an intrinsic uncertainty in the measurements.

Therefore, given the uncertainty values of the measurement typically indicated by the manufacturer of the device, the values are all pre-processed inside a block called the pre-processor of uncertainty 510. All the outputs of the pre-processor of uncertainty are fully carrying the uncertainty values. In this example vibrational data and laser data extracted are also subjected to uncertainty calculation. The main reason for this variation is due to the fact that between the laser pulses from the two lasers the vibration will be detected on the surface with a certain delay from the two interferometers. This delay may cause a small uncertainty value in the measurement of depth. Therefore that delay, or uncertainty value in the calculation, can also be taken in consideration and update the depth measurement accordingly.

Other measurements such as 3-axis IMU data with uncertainty of the other sensors, need further processing as it is required to transform all the necessary orientation, linear and angular velocities into a specific direction. This is even more important in the case of a hand-held device or a head wearable device. The 3-axis IMU data with uncertainty needs to be transformed therefore into heading via a heading processor 560. After that these 3-axis IMU values can be processed in the real-time mapper system 570. An additional sensor such as a GPS, or an RTK GPS antenna system, represented by the block 550 can be implemented to set the absolute reference frame.

In order to make sure that the images are properly reconstructed, a proper transformation via rotation matrix 580 from Real-Time Mapper to Ultrasound Reference frame may be required as shown in FIG. 5. Displacement of laser images may allow for feature detection and therefore point-to-point registration allowing for a better image formation. In addition and at later steps, outliers may also be neglected for a better performance of the calculation.

Results of the exam are shown into a 3D visualizer 592. The post-processing system related to the architecture shown in FIG. 1, FIG. 2, FIG. 3 or FIG. 4 is equipped with an information logger 591 able to record every input/output of each sensor in case the user would like to change or tune some of the sensors parameters to carry additional studies post ultrasound exam or perform different simulation and experimenting specific scenarios by modifying specific parameters. Therefore, using the Robotic Platform, makes it possible to playback the ultrasound session for that specific patient given the recording of the movement of all the external sensors and their local relative reference frames with relation to the absolute reference frame provided by the GPS. Additionally, varying vibrometers and laser orientation in the Robotic Platform can be used for assessment and prediction of alternative vibrational intensity for clinical purposes. Also, varying vibrometers and laser orientation in the Robotic Platform together with the other external sensors can be used for monitoring the future status of a tumor or even to predict if and how a tumor has the potential of progressing, shrinking or not changing. Therefore, post-processing studies can also be used to study and plan for potential therapeutic effects when administering a medication or drug and analyze different vibrational patterns.

### Example 6: Flow Chart Explaining the Post-Processing Procedure and Image Reconstruction for Two Sources Q-Switched Pulsed Laser Working Simultaneously and in Synchronization, Colored Camera, Black and White Camera, Two Interferometers Working Simultaneously and in Synchronization, GPS, 3-Axis IMU and an Additional Correction Step

Referring to FIG. 6, a flow chart setting forth the most important steps for post-processing and generating laser images is provided. To start the post-processing loop and generate 2D/3D laser images (i.e. playing back an ultrasound session using a Robotic Platform such as ROS) related to the architecture shown in FIG. 1, FIG. 2, FIG. 3 or FIG. 4, the user needs to start from where all the data from the scanning session is collected, which is the scanning session data block 640. The ultrasound lasers system scanning session data block 640 is the main container of the recorded ultrasound session. Such data are, for example, displacement, position, velocity and acceleration of every sensor and even monitoring the movements of the stepper motors. The presence of the GPS makes sure that there is an absolute reference frame from which all the other local reference frames given from the other sensors relate to while the system translates or roto-translate around the patient.

All necessary data coming from laser sources, external sensors such as color camera, black and white camera, 3-axis IMU, interferometers and a GPS, or an RTK GPS antenna system, 650 are characterized by an intrinsic uncertainty in the measurements.

Therefore, given the uncertainty values of the measurement typically indicated by the manufacturer of the device, the values are all pre-processed inside a block called the pre-processor of uncertainty 610. All the outputs of the pre-processor of uncertainty are fully carrying the uncertainty values. In this example vibrational data and laser data extracted are also subjected to uncertainty calculation. The main reason for this variation is due to the fact that between the laser pulses from the two lasers the vibration will be detected on the surface with a certain delay from the two interferometers. This delay may cause a small uncertainty value in the measurement of depth. Therefore that delay, or uncertainty value in the calculation, can also be taken in consideration and update the depth measurement accordingly.

Other measurements such as 3-axis IMU data with uncertainty of the other sensors, need further processing as it is required to transform all the necessary orientation, linear and angular velocities into a specific direction. This is even more important in the case of a hand-held device or a head wearable device. The 3-axis IMU data with uncertainty needs to be transformed therefore into heading via a heading processor 660. After that these 3-axis IMU values can be processed in the real-time mapper system 670. An additional sensor such as a GPS, or an RTK GPS antenna system, represented by the block 650 can be implemented to set the absolute reference frame.

In order to make sure that the images are properly reconstructed, a proper transformation via rotation matrix 680 from Real-Time Mapper to Ultrasound Reference frame may be required as shown in FIG. 6. Displacement of laser images may allow for feature detection and therefore point-to-point registration allowing for a better image formation. In addition and at later steps, outliers may also be neglected for a better performance of the calculation.

Results of the exam are shown into a 3D visualizer 692. The post-processing system related to the architecture shown in FIG. 1, FIG. 2, FIG. 3 or FIG. 4 is equipped with an information logger 693 able to record every input/output of each sensor in case the user would like to change or tune some of the sensors parameters to carry additional studies post ultrasound exam or perform different simulation and experimenting specific scenarios by modifying specific parameters. Therefore, using the Robotic Platform, makes it possible to playback the ultrasound session for that specific patient given the recording of the movement of all the external sensors and their local relative reference frames with relation to the absolute reference frame provided by the GPS.

Maximum vibrational surface points are located by the two interferometers and then translated into a proper grid map after compensating for rotation and scale. The particle filter algorithm can be used to predict what the next maximum vibrational point can be as it is able to find patterns in historical and current data to extend them into future predictions. In this way real-time is possible. Particle filters approximate posteriors by a finite number of values, each roughly corresponding to a region in state space. Therefore the probability (or prior) of an event occurring before new (posterior) data is collected at each detection step is how the heat grid map is formed. The posterior probability is the revised or updated probability of a surface vibrational detection event (detection of point) occurring after taking into consideration new historical information in order to predict the next vibrational point. The posterior probability is calculated by updating the prior probability. The posterior probability is the probability of event A occurring given that event B has occurred. Therefore, if during playing back an ultrasound session, a user desires to change parameters to understand the potential results and implications of a the new simulated session (e.g. changing the angle of the lasers sources emitting the pulses, duration of the pulses, angle of detection of the interferometers, flight time detection rates etc.) it is possible to understand the results applying the same particle filter techniques without having physically the patients present.

Maximum vibrational surface points are located by the two interferometers and then translated into a proper grid map after compensating for rotation and scale. The particle filter algorithm can be used to predict what the next maximum vibrational point can be as it is able to find patterns in historical and current data to extend them into future predictions. In this way real-time is possible. Particle filters approximate posteriors by a finite number of values, each roughly corresponding to a region in state space. Therefore the probability (or prior) of an event occurring before new (posterior) data is collected at each detection step is how the heat grid map is formed. The posterior probability is the revised or updated probability of a surface vibrational detection event (detection of point) occurring after taking into consideration new historical information in order to predict the next vibrational point. The posterior probability is calculated by updating the prior probability. The posterior probability is the probability of event A occurring given that event B has occurred. Therefore, if during playing back an ultrasound session, a user desires to change parameters to understand the potential results and implications of a the new simulated session (e.g. changing the angle of the lasers sources emitting the pulses, duration of the pulses, angle of detection of the interferometers, flight time detection rates etc.) it is possible to understand the results applying the same particle filter techniques without having physically the patients present. Additionally, varying vibrometers and laser orientation in the Robotic Platform can be used for assessment and prediction of alternative vibrational intensity for clinical purposes. Also, varying vibrometers and laser orientation in the Robotic Platform together with the other external sensors can be used for monitoring the future status of a tumor or even to predict if and how a tumor has the potential of progressing, shrinking or not changing. Therefore, post-processing studies can also be used to study and plan for potential therapeutic effects when administering a medication or drug and analyze different vibrational patterns.

### Example 7: Flow Chart Explaining the Real-Time Processing Procedure and Image Reconstruction for a Two Source Q-Switched Pulsed Laser Working Simultaneously and in Synchronization, Colored Camera, Black and White Camera, Two Interferometers Working Simultaneously and in Synchronization, GPS and 3-Axis IMU

Referring to FIG. 7, a flow chart setting forth the most important steps for generating laser images in real-time is provided. To start the real-time processing loop 700 and generate 2D/3D laser images related to the architecture shown in FIG. 1, FIG. 2, FIG. 3 or FIG. 4 two photoacoustics laser sources are directed toward the patient.

Laser sources 750 emit ultrasonic disturbances towards the patient, external sensors are used to precisely reconstruct the images, and are characterized by an intrinsic uncertainty in the measurements. Two external interferometers 780 and 790 are working in perfect synchronization with the two laser sources 750.

Therefore, given the uncertainty values of the measurement typically indicated by the manufacturer of the device, the values are all pre-processed inside a block called the pre-processor of uncertainty 720. All the outputs of the pre-processor of uncertainty are fully carrying the uncertainty values. The uncertainty values are therefore passed to the orientation block 730, provided by the 3-axis IMU, and the images taken by the camera resulting in the visual inertial odometry block 740. Camera images may be used to estimate the velocity of the system as it moves, calculating the displacement between sequential images and their direction and orientation in order to keep the search box area established by the doctor or the sonographer always in the field of view.

Outputs from the visual inertial odometry, 3-axis IMU and orientation are then passed to the filtering system 793 composed of four main blocks: the odometry velocity measurement block, the orientation roll-pitch-yaw measurement, position measurement and ultrasound measurement. In order to provide 2D/3D images the filtering system uses the technique of particle filtering. Real-time measurements coming from all the synchronized sensors are used to build the layer of the heat grid map at that specific depth given the penetration rate of the two lasers. In order to predict what the next measurement will be (i.e. the next depth measurement) historical and current vibrational data points are used to predict future vibrational points, providing insights into what the next depth measurement might be.

The representation of the four blocks 793 and therefore the vibrational layer (heat grid map) at a specific depth are therefore visualized in a 3D visualizer monitor 794. The real-time system related to the architecture shown in FIG. 1, FIG. 2, FIG. 3 or FIG. 4 is equipped with an information logger 795 able to record every input/output of each sensor in case the user would like to change or tune some of the sensors parameters to carry additional studies post ultrasound exam or perform different simulation and experimenting specific scenarios by modifying specific parameters. Therefore, using the Robotic Platform, makes it possible to show real-time images for that specific patient given the movement of all the external sensors and their local relative reference frames with relation to the absolute reference frame provided by the GPS. Thanks to the use of real-time prediction techniques such as the particle filter, images can be continuously updated and corrected at any step.

By way of example: Q-Switched laser A sends a pulse a time 0 at position 0 so the first wave can penetrate the surface of the skin and arrive at the preferred depth. Q-Switched laser B sends a pulse, for example, 10 nanoseconds later a time 1 at position 1 (different from the position of laser A) so the second wave can penetrate the surface of the skin and arrive at the preferred depth. Laser A emits a pulse, laser B emits a second pulse 10 nanoseconds after laser A. The first wave from laser A reaches the surface and is detected from interferometer A and 10 nanoseconds later the second wave from laser B reaches the surface and is detected from interferometer B. Every time the laser is first detected on the surface of the biological tissue, that is the moment of maximum vibrational point. That specific point is therefore associated with xyz coordinates. So in real-time, the system translates (or roto-translates) at extremely low speed within the search area defined by the doctor while it performs the scan alternating the two pulses from the two different lasers and detecting the two vibrational waves at the surface using the two different interferometers. Not only the two source Q-Switched pulsed lasers are working simultaneously and in synchronization with themselves and the two interferometers are working simultaneously and in synchronization with themselves but also the two lasers and the two interferometers are working simultaneously and in synchronization. Their perfect coordination together with the synchronization of the other external sensors are able to provide a real-time 2D/3D image.

Maximum vibrational surface points are located by the two interferometers and then translated into a proper grid map after compensating for rotation and scale. The particle filter algorithm can be used to predict what the next maximum vibrational point can be as it is able to find patterns in historical and current data to extend them into future predictions. In this way real-time is possible. Particle filters approximate posteriors by a finite number of values, each roughly corresponding to a region in state space. Therefore the probability (or prior) of an event occurring before new (posterior) data is collected at each detection step is how the heat grid map is formed. The posterior probability is the revised or updated probability of a surface vibrational detection event (detection of point) occurring after taking into consideration new historical information in order to predict the next vibrational point. The posterior probability is calculated by updating the prior probability. The posterior probability is the probability of event A occurring given that event B has occurred.

After patient A finishes an ultrasound session, the doctor has the 2D/3D heat grid map coming from interpolating all the depth layers. If patient A comes back a few months later the doctor may upload the previous heat grid map as an initial input to the 3D visualizer 794 before starting the real-time session. The doctor after that starts the new scanning session. At the end of the ultrasound session, the doctor may compare the two heat grid maps to see if any changes inside the tissue occurred and act accordingly.

### Example 8: Flow Chart Explaining the Real-Time Processing Procedure and Image Reconstruction for a Two Source Q-Switched Pulsed Laser Working Simultaneously and in Synchronization, Colored Camera, Black and White Camera, Two Interferometers Working Simultaneously and in Synchronization, GPS and 3-Axis IMU

Referring to FIG. 8, a flow chart setting forth the most important steps for generating laser images in real-time is provided. To start the real-time processing loop 800 and generate 2D/3D laser images related to the architecture shown in FIG. 1, FIG. 2, FIG. 3 or FIG. 4 two photoacoustics laser sources are directed toward the patient.

Laser sources 850 emit ultrasonic disturbances towards the patient, external sensors are used to precisely reconstruct the images, and are characterized by an intrinsic uncertainty in the measurements. Two external interferometers represented by the blocks 890 and 893 are working in perfect synchronization with the two laser sources represented by the blocks 850.

Therefore, given the uncertainty values of the measurement typically indicated by the manufacturer of the device, the values are all pre-processed inside a block called the pre-processor of uncertainty 820. All the outputs of the pre-processor of uncertainty are fully carrying the uncertainty values. The uncertainty values are therefore passed to the orientation block 830, provided by the 3-axis IMU, and the images taken by the camera resulting in the visual inertial odometry block 840. Camera images may be used to estimate the velocity of the system as it moves, calculating the displacement between sequential images and their direction and orientation in order to keep the search box area established by the doctor or the sonographer always in the field of view.

Outputs from the visual inertial odometry, 3-axis IMU and orientation are then passed to the filtering system 881 composed of four main blocks: the odometry velocity measurement block, the orientation roll-pitch-yaw measurement, position measurement and ultrasound measurement. In order to provide 2D/3D images the filtering system uses the technique of particle filtering. Real-time measurements coming from all the synchronized sensors are used to build the layer of the heat grid map at that specific depth given the penetration rate of the two lasers. In order to predict what the next measurement will be (i.e. the next depth measurement) historical and current vibrational data points are used to predict future vibrational points, providing insights into what the next depth measurement might be.

The representation of the four blocks 881 and therefore the vibrational layer (heat grid map) at a specific depth are therefore visualized in a 3D visualizer monitor 897. The real-time system related to the architecture shown in FIG. 1, FIG. 2, FIG. 3 or FIG. 4 is equipped with an information logger 898 able to record every input/output of each sensor in case the user would like to change or tune some of the sensors parameters to carry additional studies post ultrasound exam or perform different simulation and experimenting specific scenarios by modifying specific parameters. Therefore, using the Robotic Platform, makes it possible to show real-time images for that specific patient given the movement of all the external sensors and their local relative reference frames with relation to the absolute reference frame provided by the GPS. Thanks to the use of real-time prediction techniques such as the particle filter, images can be continuously updated and corrected at any step.

By way of example: Q-Switched laser A sends a pulse a time 0 at position 0 so the first wave can penetrate the surface of the skin and arrive at the preferred depth. Q-Switched laser B sends a pulse, for example, 10 nanoseconds later a time 1 at position 1 (different from the position of laser A) so the second wave can penetrate the surface of the skin and arrive at the preferred depth. Laser A emits a pulse, laser B emits a second pulse 10 nanoseconds after laser A. The first wave from laser A reaches the surface and is detected from interferometer A and 10 nanoseconds later the second wave from laser B reaches the surface and is detected from interferometer B. Every time the laser is first detected on the surface of the biological tissue, that is the moment of maximum vibrational point. That specific point is therefore associated with xyz coordinates. So in real-time, the system translates (or roto-translates) at extremely low speed within the search area defined by the doctor while it performs the scan alternating the two pulses from the two different lasers and detecting the two vibrational waves at the surface using the two different interferometers. Not only the two source Q-Switched pulsed lasers are working simultaneously and in synchronization with themselves and the two interferometers are working simultaneously and in synchronization with themselves but also the two lasers and the two interferometers are working simultaneously and in synchronization. Their perfect coordination together with the synchronization of the other external sensors are able to provide a real-time 2D/3D image.

Maximum vibrational surface points are located by the two interferometers and then translated into a proper grid map after compensating for rotation and scale. The particle filter algorithm can be used to predict what the next maximum vibrational point can be as it is able to find patterns in historical and current data to extend them into future predictions. In this way real-time is possible. Particle filters approximate posteriors by a finite number of values, each roughly corresponding to a region in state space. Therefore the probability (or prior) of an event occurring before new (posterior) data is collected at each detection step is how the heat grid map is formed. The posterior probability is the revised or updated probability of a surface vibrational detection event (detection of point) occurring after taking into consideration new historical information in order to predict the next vibrational point. The posterior probability is calculated by updating the prior probability. The posterior probability is the probability of event A occurring given that event B has occurred.

The additional block 882 shows that images properly processed may be subjected to an additional matching step where outside stereo images taken using the cameras overlap with the vibrometric images (grid heat map) optically detected at that depth using the interferometer. So any vibrational point outside of the search area can be considered an outlier and, therefore, neglected in real-time.

After patient A finishes an ultrasound session, the doctor has the 2D/3D heat grid map coming from interpolating all the depth layers. If patient A comes back a few months later the doctor may upload the previous heat grid map as an initial input to the 3D visualizer 794 before starting the real-time session. The doctor after that starts the new scanning session. At the end of the ultrasound session, the doctor may compare the two heat grid maps to see if any changes inside the tissue occurred and act accordingly. The main reason of the block 880 would be that between the two ultrasound sessions, the system in FIG. 1, FIG. 2, FIG. 3 or FIG. 4 could have been subjected to recalibration. Therefore, making sure that the reference frames are oriented the same is important to give the correct interpretation to the heat grid map images. To be specific the reference frames form the ultrasound session A for patients A taken at a specific location need to be the same reference frame for session B for the same patients to be able to property overlap the heat grid map and assess the difference in biological tissues. Therefore, if necessary, an additional recalibration step to align the old ultrasound reference frame taken during session A can be applied to the new reference frame and compensate for any misalignments or incorrect alignments.

It would be appreciated by those skilled in the art that various changes and modifications can be made to the illustrated embodiments without departing from the spirit of the present invention. All such modifications and changes are intended to be within the scope of the present invention except as limited by the scope of the appended exemplary claims.

While there is shown and described herein certain specific structure embodying the invention, it will be manifest to those skilled in the art that various modifications and rearrangements of the parts may be made without departing from the spirit and scope of the underlying inventive concept and that the same is not limited to the particular forms herein shown and described except insofar as indicated by the scope of the appended claims.

### LIST OF EMBODIMENTS AND EXAMPLES

Specific systems and methods of obtaining laser ultrasound images completely contactless have been described. The detailed description in this specification is illustrative and not restrictive or exhaustive. The detailed description is not intended to limit the disclosure to the precise form disclosed. Other equivalents and modifications besides those already described are possible without departing from the inventive concepts described in this specification, as those skilled in the art will recognize. When the specification or claims recite methods, steps or functions in order, alternative embodiments may perform the tasks in a different order or substantially concurrently. The inventive subject matter is not to be restricted except in the spirit of the disclosure.

When interpreting the disclosure, all terms should be interpreted in the broadest possible manner consistent with the context. Unless otherwise defined, all technical and scientific terms used in this specification have the same meaning as commonly understood by one of ordinary skills in the art to which this invention belongs. This invention is not limited to the particular methodology, systems, protocols, and the like described in this specification and, as such, can vary in practice. The terminology used in this specification is not intended to limit the scope of the invention, which is defined solely by the claims.

What is claimed is:

1. A system comprising of:
a) two photoacoustic laser excitation sources working simultaneously and in synchronization with the same wavelength to direct ultrasonic waves into a desired area of tissue distributing acoustic energy into the tissue at the speed of sound;
b) two optical interferometers working simultaneously and in synchronization to detect surface vibrations on the area of interest;
c) a data acquisition system configured to process data originating from the backscattering of ultrasonic waveform sources on the surface of the tissue;
d) a processor system configured to detect ultrasonic waveform sources to assess an internal structure of organs and tissues;
e) a robotic platform configured to include the two laser sources working simultaneously and in synchronization, two optical interferometers working simultaneously and in synchronization, the GPS, the color camera, the black and white camera and the stepping motors used for translation and roto-translation;
f) a 3-axis IMUs mounted on each laser, optical interferometer, GPS, color camera, black and white camera;
g) and two focused compensation lenses for directing the photoacoustic excitation laser sources into the tissue or the area of interest.

2. A system comprising of:
a) two photoacoustic laser excitation sources working simultaneously and in synchronization with the same wavelength to direct ultrasonic waves into a desired area of tissue at the speed of sound;
b) two optical interferometers working simultaneously and in synchronization to detect surface vibrations on the area of interest;
c) two photoacoustic laser excitation sources working simultaneously, in conjunction with one another, and in synchronization with minimal wavelength displacement in order to form laser stereometry images;
d) a data acquisition system configured to process data originating from the backscattering of ultrasonic waveform sources on the surface of the tissue;
e) a processor system configured to detect ultrasonic waveform sources to assess an internal structure of organs and tissues;
f) a robotic platform configured to include the two laser sources working simultaneously and in synchronization, two optical interferometers working simultaneously and in synchronization, the GPS, the color camera, the black and white camera and the stepping motors used for translation and roto-translation;
g) a 3-axis IMUs mounted on each laser, optical interferometer, GPS, color camera, black and white camera and the stepping motors used for translation and roto-translation;
h) and a roto-translation apparatus that translates and rotates around the patient.

3. A system comprising of:
a) two photoacoustic laser excitation sources working simultaneously, in conjunction with one another, and in synchronization with wavelength difference in order to form laser stereometry images at different depth at the same time and in synchronization to direct ultrasonic waves into a desired area of tissue distributing acoustic energy into the tissue at the speed of sound;
b) two optical interferometers working simultaneously and in synchronization to detect surface vibrations on the area of interest;
c) a data acquisition system configured to process data originating from the backscattering of ultrasonic waveform sources on the surface of the tissue;
d) a processor system configured to detect ultrasonic waveform sources to assess an internal structure of organs and tissues;
e) a robotic platform configured to include the two laser sources working simultaneously and in synchronization, two optical interferometers working simultaneously and in synchronization, the GPS, the color camera, the black and white camera and the stepping motors used for translation and roto-translation;
f) a 3-axis IMUs mounted on each laser, optical interferometer, GPS, color camera, black and white camera;
g) and two focused compensation lenses for directing the photoacoustic excitation laser sources into the tissue or the area of interest.

4. A system comprising of:
h) two photoacoustic laser excitation sources working simultaneously, in conjunction with one another, and in synchronization with wavelength difference in order to form laser stereometry images at different depth at the same time and in synchronization to direct ultrasonic waves into a desired area of tissue distributing acoustic energy into the tissue at the speed of sound;
i) two optical interferometers working simultaneously and in synchronization to detect surface vibrations on the area of interest;
j) a data acquisition system configured to process data originating from the backscattering of ultrasonic waveform sources on the surface of the tissue;
k) a processor system configured to detect ultrasonic waveform sources to assess an internal structure of organs and tissues;
l) a robotic platform configured to include the two laser sources working simultaneously and in synchronization, two optical interferometers working simultaneously and in synchronization, the GPS, the color camera, the black and white camera and the stepping motors used for translation and roto-translation;
m) a 3-axis IMUs mounted on each laser, optical interferometer, GPS, color camera, black and white camera and the stepping motors used for translation and roto-translation;
n) and a roto-translation apparatus that translates and rotates around the patient.

5. A method for generating real-time laser ultrasound 2D-3D images of a subject comprised of:
a) drawing specific points on the skin of the patient with a highlighter and connect them allowing the external color camera and black and white camera to feature-detect those points and establish a specific search area box;
b) two photoacoustics excitation sources working simultaneously, in conjunction with one another and in synchronization with minimal wavelength displacement range emitting ultrasonic waves into a tissue within the search area box determined by the doctor;
c) using two optical interferometers working simultaneously and in synchronization to detect the maximum vibrational points on the surface of the tissue within the search area box determined by the doctor by assigning xyz coordinates to every point;
d) extracting the maximum vibrational intensity for every detected surface point;
e) assigning the cartesian coordinates to each vibrational intensity point detected;
f) combining all the vibrational coordinate points together to create a heat grid map at a specific depth according to the penetration power of the two photoacoustic excitation sources;
g) interpolating the different layers to form a completely contactless 3D image;
h) applying particle filtering techniques to predict a 2D/3D image at another specific depth;
i) visualizing the 2D-3D reconstructed images into a proper visualization processor system; and
j) saving sensor data positions, orientation, displacement, velocities and vibrational points to a data logger.

6. The method of claim 5, wherein constructing a vibrational map comprises calculating the time arrival difference between two vibrational waves to identify the two vibrational maximum intensity points and extract the depth measurement.

7. The method of claim 5, wherein additional vibrational layers can be obtained varying the penetration power of the two photoacoustic excitation sources within the search box defined by the doctor.

8. The method of claim 5, wherein the model of the body or the search area box is obtained using camera stereometry techniques from multiple views of the entire body.

9. The method of claim 5, wherein the vibrational layer of the body or the search area box is obtained using laser stereometry techniques.

10. A method for generating post-processing laser ultrasound 2D-3D images of a subject comprised of:
a) accessing the Ultrasound Laser System Scanning Session Data that contains all the sensors information obtained from a real-time session previously performed;
b) opening on a Robotic Platform the Ultrasound Laser System Scanning Session Data;
c) preparing the proper local reference frames of the external sensors in the Robotic Platform;
d) preparing the proper absolute reference frames of the external gps sensor in the Robotic Platform;
e) combining all local and absolute reference frame of the external sensors and gps to obtain precise location in the Robotic Platform;
f) playing all the time stamped sensors information from where they started working at the beginning of the real-time session to where they stopped working during the real-time session;
g) interpolating the different information from the optical interferometers to obtain a completely contactless 3D image;
h) applying particle filtering techniques to predict a 2D/3D image from the data present in the Ultrasound Laser System Scanning Session Data;
i) visualizing the 2D/3D reconstructed images into a proper visualization processor system;
j) and saving sensor data positions, orientation, displacement, velocities and vibrational points predicted to a data logger.

11. The method of claim 10, wherein varying sensor information and orientation in the Robotic Platform can be used for studying purposes.

12. The method of claim 10, wherein varying vibrometers and laser orientation in the Robotic Platform can be used for assessment and prediction of alternative vibrational intensity for clinical purposes.

13. The method of claim 10, wherein varying vibrometers and laser orientation in the Robotic Platform can be used for monitoring the future status of a tumor.
